(19) United States
(12) Patent Application Publication
(10) Pub. No.: US 2005/0133042 A1
(43) Pub. Date: Jun. 23, 2005

(54) Neck collars for relieving neck pains

(76) Inventor: Bashir Zikria, Norwood, NJ (US)

Correspondence Address: EVELYN M. SOMMER, 250 PARK AVE, RM 825, NEW YORK, NY, 10221, US

(21) Appl. No.: 10/737,885

(22) Filed: Dec. 18, 2003

(51) Int. Cl. A61F 5/00

(52) U.S. Cl. 128/845; 128/846

(57) ABSTRACT

A method of relieving neck, upper back, shoulder and arm pain due to injury, degenerative and inflammatory diseases, tumors, infections or congenital abnormalities of the vertebrae by having the subject having such pain wear a neck collar which is held securely around the neck and under the wearer's chin that permits lateral head motion but prevents downward flexion of the head.

This application is a related to application Ser. No. 08/967562 filed Nov. 12, 1997, and issued to U.S. Patent.

### BACKGROUND OF THE INVENTION

The present invention relates, in general, to neck collars and, particularly, neck collars that reduce neck, shoulder and back pains, such as cervical osteoarthritic neck pains, cervical “pinched nerve syndrome” and shoulder and interscapular pains, by maintaining the head of the user in an almost erect position to relieve the muscular and spinal stresses. More particularly the invention relates to a method for relieving neck, shoulder and back pain by having the subject having such pain wear a neck collar which is held securely around the neck and under the wearer's chin which permits lateral head motion but prevents downward flexion of the head.

The neck (cervical spine) is composed of vertebrae, which begin in the upper torso and end at the base of the skull. The bony vertebrae along with the ligaments provide stability to the spine. The muscles allow for support and motion. Because the neck is less protected than the rest of the spine, the neck can be vulnerable to injury and disorders that produce pain and restrict motion.

Neck pain affects from 12 to 34 percent of the normal population (U.S. Pat. No. 6,599,257). Most neck pains are caused by maintaining one's head in an awkward position for too long and consequently, joints and muscles become fatigued. These minor, posture-related neck pains frequently disappear on their own after rest. More severe neck pains may result from abnormalities in the soft tissues, the muscles, ligaments, and nerves-as well as in the vertebrae and discs of the spine. The most common causes of neck pains are soft tissue abnormalities due to injury or prolonged wear and tear, or congenital malformation. Neck problems may be the source of pain in the upper back, shoulders, arms, fingers or legs.

Various degenerative and inflammatory diseases, such as cervical osteoarthritis and rheumatoid arthritis, might cause neck pains.

Osteoarthritis is a disease of the joints and is the most common form of arthritis and afflicts more than 20 million American adults. Osteoarthritis is caused by the breakdown of cartilage, the substance that provides a cushion between the bones of the joints. The excessive wear of cartilage causes the bones under the cartilage to rub together, causing pain, swelling and loss of motion in the joint. Though the exact causes of osteoarthritis are not known, it tends to be more prevalent among people over the age of 45, female, those who suffered joint injuries from sports, work-related activities or accidents, obesity, and those who suffer from certain diseases that affect the structure and function of cartilage, such as rheumatoid arthritis, hemochromatosis, Paget's disease and gout. With 35% of Americans being overweight and an additional 27% being obese (National Health and Nutrition Examination Survey 1999, Center of Disease Control and Prevention), arthritis is expected to afflict more people in the near future.

In particular, cervical osteoarthritis, also known as arthritis of the neck, cervical spondylosis, or degenerative joint disease of the neck, is a degenerative process that affects the cervical spine, mainly affecting older people, usually over 40 and more men than women. Stiffness and pain in the neck or in the lower back can result from cervical osteoarthritis. Some people might also experience numbness, weakness or pain in the arms, hands, fingers or legs, as well as possible problems with urination.

Spinal stenosis causes the foramen, small neural passageways, to narrow and possibly compress and entrap nerve roots, therefore causing neck, shoulder, and arm pain and numbness when these nerves are unable to function properly.

Cervical radiculopathy, also known as “pinched nerve” is one type of damage or injury to a nerve or set of nerves from compression, constriction, or stretching, with consequences that can range from aches and pains to a loss of feeling or weakening of muscles. Roughly 85 in every 100,000 people will suffer from it (U.S. Pat. No. 6,539,560). One common cause of the nerve irritation is the damage to a spinal disk from injury or degenerative processes. The damaged disk can tear, allowing the soft jelly-like center to bulge out and press on an adjacent nerve. This condition tends to occur in the low back (lumbar spine) and neck (cervical spin). Heavy lifting, obesity and contact sports are some of the contributing factors to this problem.

Cervical disk degeneration also can cause neck pain. In cervical disk degeneration, the normal gelatin-like center of the disk degenerates and the space between the vertebrae narrows. As the disk space narrows, added stress is applied to the joints of the spine causing further wear and degenerative disease. The cervical disk may also protrude and cause pressure on the spinal cord or nerve roots when the rim of the disk weakens. This condition is known as herniated cervical disk.

Neck pain can also be caused by cervical strain, which is an acute or chronic inflammation of the muscles of the neck and upper shoulder area. Cervical strain can result from poor head or shoulder positioning when sleeping or during daily activities, repetitive use of arms overhead, heavy lifting, or trauma. Symptoms of cervical strain might include neck and/or upper shoulder pain, reduced neck or shoulder motion, decreased strength and headaches.

Neck pain can also be caused by injuries, such as those resulting from motor vehicle or diving accidents, contact sports, and falls. Most commonly, the neck injuries are to the soft tissues, i.e., muscles and ligaments, but in severe cases, the bones in the neck can be fractured or dislocated. One of the typical injuries is called whiplash, which is an acute cervical ligament sprain and/or muscle strain that occurs in athletes or those involved in car accidents. Whiplash occurs when a sudden, forceful motion causes the head to move in a forward then backward motion.

Depending on the diagnosis, most patients with neck pains can be treated successfully with rest, medication, immobilization, physical therapy, exercise, activity modifications, weight control or a combination of these methods. If the neck pain is caused by inflammation as a result of stretching muscles and ligaments beyond their limits, typical prescriptions might include rest and a soft neck collar, as well as medications to relieve inflammation.

Traction therapy or proper stretching of the neck and cervical spin regions can be employed to relieve the neck pains and related headaches, including those cases due to deterioration of cervical disks, pinched nerves, or lack of full range of motion in the neck. U.S. Pat. No. 6,447,468 teaches a portable traction device for cervical spine that can be worn without professional assistance and without limiting normal activity. U. S. Pat. No. 5,024,214 teaches a traction device to alleviate the neck and/or back pain and requires the user to be in a recumbent position.

U.S. Pat. No. 6,539,560 teaches a head support device, which can support the complete weight of the skull by the pads placed over the mastoid process, and therefore reduce the possibility of “pinched nerve” when the head is in hyper-extended position.

U.S. Pat. No. 6,599,257 teaches a cervical therapy device, which is capable of providing continuous motion to the cervical spine in a consistent or repetitive manner to alleviate the neck pain.

U.S. Pat. Nos. 5,336,138 and 5,116,359 teach head, neck and shoulder exercise devices to alleviate the pains associated with those body parts.

U.S. Pat. Nos. 3,572,328; 5,097,824; 5,141,489; 5,180,361 and 5,215,517, in addition to many others, teach the construction of cervical collars, which can be used to support head and neck to alleviate neck pains due to injuries and other conditions, such as whiplash.

It is an object of this invention to provide an apparatus that can reduce the neck pain by maintaining the chin markedly above the user's chest.

It is a further object of this invention to provide a flexible device for the relief of neck, shoulder and back pains.

Yet a further object of this invention is to provide such advantageous functions without unduly or unnecessarily restricting other normal movement of the head or neck to thereby maximize the comfort and acceptability of the apparatus and permit normal activity and sleep.

### SUMMARY OF THE INVENTION

The present invention provides a device for the relief of neck, shoulder and back pains. The present invention provides such a pain-relieving device, which is held securely around and under the neck and under the chin and which is simple to construct and reliable in its use.

The apparatus comprises a shaped body which when worn, lies over the anterior of a user's neck. Because the apparatus is to be worn during both regular activity and sleep, the shaped body has a cross-sectional diameter that allows free side-to-side movements of the head. Despite the unrestricted lateral head motion, the device of the present invention prevents downward flexion of the head onto the user's chest. The shaped body has a first and second lateral end. Extending from each end is a connecting cord or strap. These cords or straps attach to each other at the posterior of the user's neck by a conventional means such as, for example by a hook-and-loop system, snaps or ties. As a result, the straps maintain the position of the shaped body between the first end and second end beneath the user's chin, and the shaped body in this position maintains the user's neck in an extended position. In one embodiment of the present invention, neck straps are provided which are secured together in a way that adjusts to the diameter of the user's neck.

In another embodiment of the present invention, the apparatus is a largely flexible collar having a rigid core covered with a soft, absorbent fabric. The rigid core can be made from Styrofoam.

Each attachment strap has a first proximal end attached to the cylindrical body. In a preferred embodiment, the second or distal end of each strap has a hook-and-loop fastening material attached thereto. When the straps are attached together, the pain-relieving apparatus is maintained securely and comfortably beneath the user's chin. By elevating the chin from the chest, head slumps or drooping can be prevented and therefore the consequent excessive stress placed on the muscles, and spine can be removed to alleviate the neck pain.

### BRIEF DESCRIPTION OF THE DRAWINGS

FIG. 1 is a front elevational view of an embodiment of the pain-relieving device of the present invention;

FIG. 2 is a front elevational view of a user wearing the pain-relieving device of the present invention;

FIG. 3 is a front view of an alternative embodiment of the shaped body of the pain-relieving device of the present invention;

FIG. 4 is a front lower view of a further alternative embodiment of the shaped body of the pain-relieving device of the present invention showing the neck straps; and

FIG. 5 is a front elevational view of still further alternative embodiment of the shaped body of the pain-relieving device of the present invention showing the neck straps.

### DETAILED DESCRIPTION OF THE INVENTION

While this invention is susceptible of embodiment in many different forms, there is shown in the drawings and will herein be described in detail, several specific embodiments with the understanding that the present disclosure is to be considered as an exemplification of the principles of the invention and is not intended to limit the invention to the embodiments illustrated.

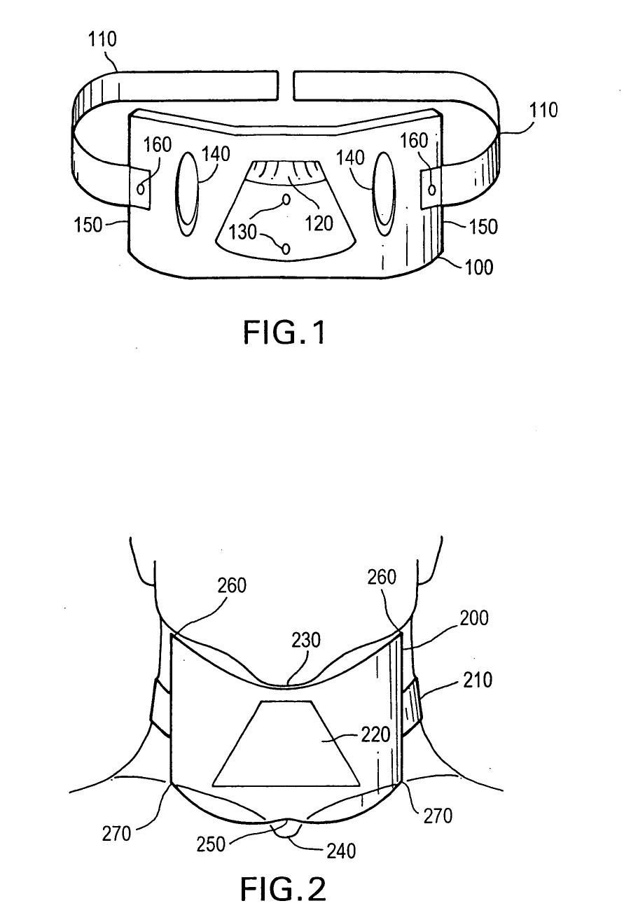

A preferred embodiment of the present invention is shown in FIG. 1 where the pain-relieving device has a shaped body 100 with a rigid chin brace 120 desirably molded into the shaped body. At each end of the shaped body, 150, straps, or cords, 110, are attached to the shaped body. The distal end of each of the straps has a connecting means that allows the straps to be adjustably secured together so that the straps can secure the shaped body in place about the user's neck.

In another embodiment of the present invention, shaped body 100, is a conventional soft cervical collar, approximately 1 inch thick, in which a chin brace 120 is secured in place by a conventional means, such as adhesive, sewing, rivets or clips such as plastic clips at 130. Typically, straps 100 are secured to the shaped body 100 in a similar fashion at 160.

In a further embodiment of the present invention, the shaped body 100 has a plurality of windows 140 that reduce the surface area of the neck that is covered by the shaped body without reducing the ability of the pain-relieving device of the present invention to restrain forward cervical vertebral flexion. Windows 140 can also reduce the weight of the pain-relieving device of the present invention thereby improving the user's comfort when wearing the device of the present invention.

Chin brace 120 can be made of any material that maintains the chin position and is compatible with prolonged contact with the skin. Plastic is a preferred chin brace material and polyvinyl chloride, polyethylene, styrene acrylonitile are particularly preferred plastics for use as chin brace material in the present invention.

It is also preferred that the chin brace is made of a size, length and width, that conforms to the user's neck. Typically, the chin brace is about 0.125 inches thick and curved to rest under the user's chin at an angle of between about 120 and 145 degrees. neck brace such as that sold as the Philadelphia™ Cervical Collar, is secured to straps and the distal end of the straps have a conventional connecting means that allow the cervical collar front portion to be secured under the user's chin and restrain forward cervical vertebral flexion.

FIG. 2 illustrates the use of the pain-relieving device of the present invention. The shaped body 200 is secured by straps 210 about the neck of the user with chin brace 220 restraining the user's chin from forward cervical vertebral flexion. In use, the distance from the top center of the shaped body, 230, to the bottom of the shaped body directly under position 230, should be effective to extend the tip of the chin to the manubrium to the users fullest comfortable neck extension. Furthermore, it is preferred that the distance from the top to the bottom of the shaped body between the mid-mandible, 260, and the mid-clavicle, 270, conforms to the distance when the user's neck is in its fullest comfortable extension.

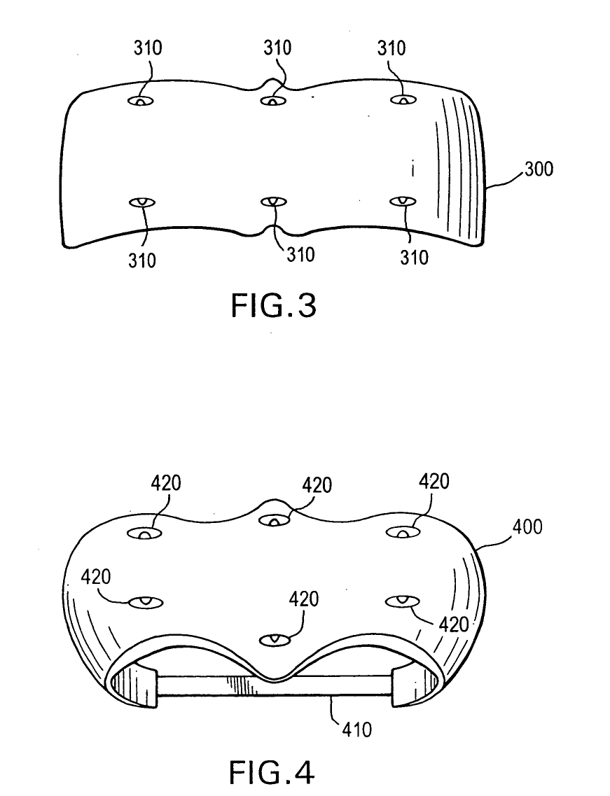

FIG. 3 shows an alternative embodiment of the shaped body 300 of the present invention. In this embodiment, the shaped body 300 is a modified soft cervical collar having an approximately one-inch thickness and a plurality of rigid struts 310. Plastic is a preferred material for making the struts, although the struts 310 can be made of any suitable material such as a lightweight metal or a wood. In a preferred version of this embodiment, there are three struts 310 rigidifying the otherwise soft cervical collar. These struts are effective to allow the collar to restrain the user's forward cervical vertebral flexion and keep the user's neck in its fullest comfortable extension. In the embodiments of the pain-relieving device of the present invention that use struts, it is preferred that the struts are secured in place by a conventional means such as adhesive, clips or rivets. Typically, the strut or struts in the center of the collar in an embodiment using struts are between about 4 and 6 inches in length and the struts in the mid-mandible area are between about 3 and 5 inches in length.

FIG. 4 shows yet another alternative embodiment of the present invention with shaped body 300 in yet another conformation. In this embodiment, shaped body 400 is again a soft cervical collar rigidified by a plurality of struts 420, Additionally, this FIG. 4 shows strap 410 that secures the collar about the user's neck.

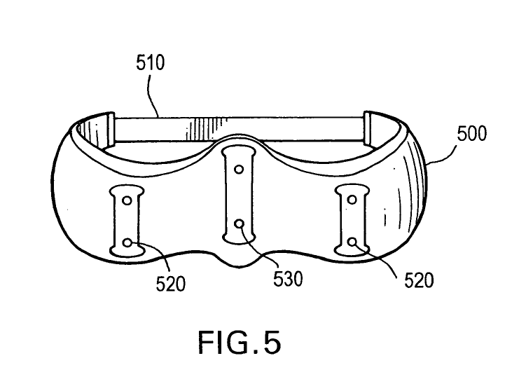

FIG. 5 illustrates yet a further alternative embodiment of the present invention. Shaped body 500 is secured about the user's neck by straps 510 and has a plurality of struts, 520 and 530 that rigidify the otherwise soft collar so as to restrain the user's forward cervical vertebral flexion. The length of strut 530 is such that it maintains the user's neck in its fullest comfortable extension. Struts 520 are such that they ensure that the collar between the mid-mandible and the mid-clavicle conforms to distance that exists with the user's neck in its fullest comfortable extension.

While the invention has been illustrated and described as embodied in an improved pain-relieving device, it is not intended to be limited to the details shown, since it will be understood that various omissions, modifications, substitutions and changes in the forms and details of the device illustrated and in its operation can be made by those skilled in the art without departing in any way from the spirit of the present invention Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. Method for treating neck, shoulder and back pain, which comprises applying securely around and under the neck of a subject in need of such treatment a pain-relieving device comprising
a) shaped body having therein a chin brace;
b) attached to each lateral end of said shaped body, a strap, said straps having connecting means effective to secure said chin brace in said shaped body under the wearer's chin so as to restrain the wearer's neck in a position that is extended substantially as much as possible without discomforting said wearer and without substantially restricting the lateral motion of the wearer's head

2. Method according to claim 1 wherein said pain-relieving device has a plurality of windows

3. Method according to claim 1 wherein said pain-relieving device rests under said wearer's chin at an angle between about 120 and 145 degrees.

4. Method for treating neck, shoulder and back pain which comprises applying securely around and under the neck of a subject in need of such treatment a pain-relieving device comprising
a) a shaped body having therein a plurality of struts;
b) attached to each lateral end of said shaped body, a strap, said straps having a connecting means effective to secure said shaped body under the wearer's chin so as to restrain the wearer's neck in a position that is extended substantially as much as possible without discomforting said wearer.
